(19) United States
(12) Patent Application Publication
(10) Pub. No.: US 2013/0041270 A1
(43) Pub. Date: Feb. 14, 2013

(54) BLOOD PRESSURE MEASURING DEVICE AND SYSTEM WITH AUTOMATIC SELF-EXAMINATION AND SELF-CALIBRATION FUNCTIONS

(71) Applicant: Kuo-Yuan Chang, New Taipei City (TW)

(72) Inventor: Kuo-Yuan Chang, New Taipei City (TW)

(21) Appl. No.: 13/371,827

(22) Filed: Feb. 13, 2012

(30) Foreign Application Priority Data
Aug. 8, 2011 (TW) 100128106

(51) Int. Cl. A61B 5/021

(52) U.S. Cl. 600/485

(57) ABSTRACT

A blood pressure measuring system with automatic self-examination and self-calibration functions, which utilizes an examining and calibrating unit to automatically compare pressure information detected by a signal detection unit with an original factory calibration value for pressure, to self-calibrate the blood pressure measuring system, wherein the examining and calibrating unit can be arranged in a blood pressure measuring device or in an external electronic device, for providing users a method to confirm accuracy of the blood pressure measuring system to prevent serious consequence caused by miscalculations of blood pressure and physiological values. Accordingly, original factory calibration can be skipped to save time and money; calibration and examination processes are simplified and more convenient.

### BACKGROUND OF THE INVENTION

1. Field of the Invention

The present invention relates to a blood pressure measuring device and system, and more particularly to a blood pressure measuring device and system with automatic self-examination and self-calibration functions.

2. Description of the Prior Art

In recent years, population ageing and low birth rate have gradually become a trend worldwide. In addition, prevalence of lifestyle disease for modern people dramatically increases especially for middle-aged and elderly people because quality of material life is improved. According to the statistics of Bureau of Health Promotion, Department of Health, R.O.C. (Taiwan), there is one hundred thousand of hypertension patients increased every year, that is, 274 patients increased per day in Taiwan. So far the population of hypertension amounts to 4 million; prevalence rate of hypertension among people who are 65 years old and above particularly reaches to 56.6%. It can be seen that there are numerous people in high risk group for cardiovascular disease. If blood pressure cannot be well-controlled, it may induce lesions or complications to patients' brains, kidneys, eyes and so on. To make matters worse, it can be fatal. Therefore, accuracy of a blood pressure measuring device is of great importance in modern society.

Most sphygmomanometers available in the market have measuring, calculating and displaying functions. When finishing measurement, it can calculate and display the results instantly. These devices are unable to self-examine and self-calibrate measurement results and should be returned for factory calibration after being used for a period of time.

Generally speaking, in the region with distinct temperature difference, for example, Northeast China with outdoor temperature of −27° C., circuit boards inside electronic sphygmomanometers which are placed outdoors may deform and be distorted owing to temperature difference. When these devices are placed indoors, temperature will be restored to 18° C. and the circuit boards inside will be distorted again in the recovery process. It results in inaccuracy in measurement results. In addition, electronic sphygmomanometers are easy to use and carry, for the reason of accuracy in measurement, devices are usually calibrated at factories' altitudes with special instruments before shipping. When users move to high altitudes, deviation occurs to internal calibration values of the devices because of altitude difference. It leads to inaccuracy to results measured at high altitudes. The Sphygmomanometers must then be returned back for factory calibration. The process is troublesome and inconvenient to hospitals and general users who are unable to ensure accuracy by themselves at home. Besides, money, time and manpower wasted on returning sphygmomanometers to the original factory are great burdens to users who have to control their blood pressure values regularly.

General electronic sphygmomanometers without automatic self-examination and self-calibration functions are widely used over hospitals, clinics and home care centers. Long term use or alternating temperature can lead to inaccuracy to blood pressure measuring devices; doctors may dose patients with wrong drugs. When nursing staff discovers measurement deviation during regular use or annual maintenance, devices must be returned back for factory calibration. For departments which have to use sphygmomanometers every day, time-wasting process for returning devices to original factory and deviation in measuring blood pressure of patients are serious problems.

General personal use electronic sphygmomanometers cannot automatically self-examine and self-calibrate for accuracy. Long-time use or alternating temperature can lead to inaccuracy in measurement and cause deviation to measuring blood pressure without knowing it. If the devices are returned for factory calibration regularly, time and money wasted on it is quite troublesome to users who must control blood pressure every day or to users who take medication according to measurement results of blood pressure.

Currently electronic sphygmomanometers are widely used by telecare centers to measure and transmit information of blood pressure and physiological values of patients back to the centers or hospitals. Measurement deviation generated from long-time use or alternating temperature to blood pressure measurement is a serious problem. Money and time wasted on process for factory calibration are quite inconvenient to nursing centers which have to provide service to measure blood pressure and physiological values for community residents.

Deviation occurring from long-time use or alternating temperature to blood pressure measurement is a serious problem to people who need personal health care at home or personal telecare. Money and time wasted on process for returning the sphygmomanometers back for factory calibration is quite troublesome to users who must control their blood pressure everyday or who rely on blood pressure measurement for taking medication.

Accordingly, it is highly desirable to develop a blood pressure measuring system which can self-examine and self-calibrate for accuracy and is easily-operated.

### SUMMARY OF THE INVENTION

The present invention relates to a blood pressure measuring system with automatic self-examination and self-calibration functions. This blood pressure measuring system can utilize its calibration function for accuracy. There is no need for factory calibration with special instruments and extra examination. This system does not raise the cost of the products and calibration can be achieved without extra expense and manpower.

The present invention is adapted for separating power supply, display, and calculation processing units to an external electronic device, such as an electrically connected personal computer, a personal digital assistant, a set of mobile communication equipment, or a database. Therefore, electronic components of blood pressure measuring devices can be simplified. A long-time and substantial physiological information process and monitoring can be executed, and the information and calibrated values can be transmitted to a server system for further integration and process, so that a proper assistance can be provided to patients, elderly people and general users to meet their unique requirements.

In order to achieve objectives aforementioned, in one embodiment of the present invention, a blood pressure measuring system with automatic self-examination and self-calibration functions includes: a signal detection unit, for detecting an analogous signal of pressure information; a signal transformation unit connected with the signal detection unit, for receiving the analogous signal and transform it to a digital signal of pressure value; an examining and calibrating unit, for automatically comparing at least one of the analogous signal and the digital signal of pressure value with an original factory calibration value for pressure so as to self-calibrate the blood pressure measuring system; and a calculation processing unit connected to the examining and calibrating unit, the signal detection unit, and the signal transformation unit so as to calculate physiological values including blood pressure according to at least one of the analogous signal and the digital signal of pressure value.

In another embodiment of the present invention, a blood pressure measuring system with automatic self-examination and self-calibration functions is composed of a blood pressure measuring device with automatic self-examination and self-calibration functions, which is electrically connected to an external electronic device. The blood pressure measuring device with automatic self-examination and self-calibration functions includes: a signal detection unit, for detecting an analogous signal of pressure information; a signal transformation unit connected to the signal detection unit, for receiving the analogous signal and transform it to a digital signal of pressure value, a calculation processing unit with a built-in original factory calibration value for pressure of a blood pressure measuring system; and a connecting unit electrically connected to the signal transformation unit and the calculation processing unit, for connecting to an external electronic device so as to exchange the digital pressure value and the original factory calibration value for pressure.

The objective, technologies, features and advantages of the present invention will become more apparent from the following description in conjunction with the accompanying drawings, wherein certain embodiments of the present invention are set forth by way of illustration and examples.

### BRIEF DESCRIPTION OF THE DRAWINGS

The foregoing aspects and many of the accompanying advantages of this invention will become more readily appreciated as the same becomes better understood by reference to the following detailed description, when taken in conjunction with the accompanying drawings, wherein:

FIG. 1 is a linear diagram illustrating original factory calibration values for blood pressure versus digital signals;

FIG. 2 is a schematic diagram illustrating deviation for blood pressure versus digital signals;

FIG. 3. is a schematic diagram illustrating structure of a blood pressure measuring system with automatic self-examination and self-calibration functions according to an embodiment of the present invention;

FIG. 4 is a schematic diagram illustrating architecture flowchart of a blood pressure measuring system with automatic self-examination and self-calibration functions according to an embodiment of the present invention; and

FIG. 5 is a schematic diagram illustrating the structure of a blood pressure measuring system with automatic self-examination and self-calibration functions according to another embodiment of the present invention.

### DESCRIPTION OF THE PREFERRED EMBODIMENT

The detailed description is provided below and the preferred embodiments described are only for the purpose of description rather than for limiting the present invention.

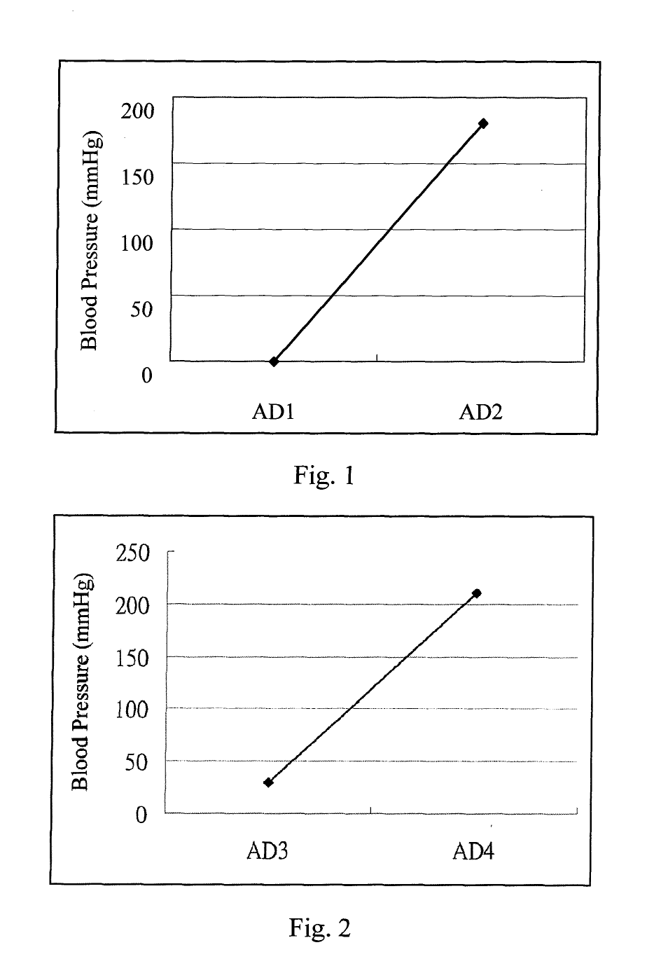

The present invention proposes a blood pressure measuring system with automatic self-examination and self-calibration functions 10 for improving current market available blood pressure measuring devices. For general blood pressure measuring system, the system reacts according to different pressure values, for example, sampling pressure 0 mmHg. However, for the present invention, as shown in FIG. 1, digital signal values and the corresponding pressure values (indicated as AD1, AD2) are original factory calibration values which are built in the blood pressure measuring system with automatic self-examination and self-calibration functions. As shown in FIG. 2, the original factory calibration values of the linear diagram can be used to compare with pressure values detected (for example, 0 mmHg) when booting the system or at specific time so as to obtain measuring deviation of pressure values, and correct the measuring deviation.

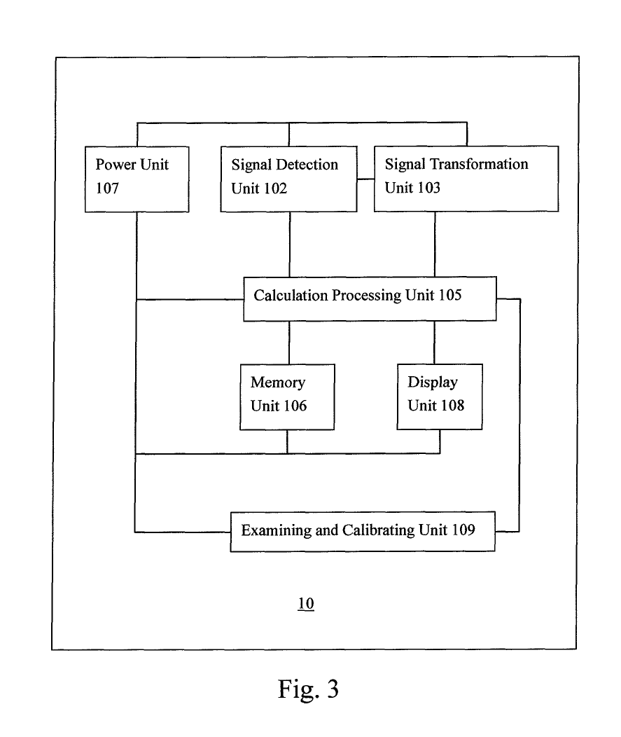

FIG. 3 shows a schematic diagram for a blood pressure measuring system with automatic self-examination and self-calibration functions. The blood pressure measuring system 10 includes a signal detection unit 102, for detecting an analogous signal of pressure information; a signal transformation unit 103, connected to the signal detection unit 102 so as to receive the analogous signal and transform it to a digital signal of pressure value; an examining and calibrating unit 109, for automatically comparing the analogous signal or digital pressure value with an original factory calibration value for pressure so as to self-calibrate the blood pressure measuring system 10; and a calculation processing unit 105, connected to the examining and calibrating unit 109, the signal detection unit 102 and the signal transformation unit 103 to calculate physiological values according to the analogous signal or the digital signal of pressure value. In one embodiment of the present invention, the physiological values include a blood pressure value.

Following the above descriptions, the blood pressure measuring system with automatic self-examination and self-calibration functions 10 further comprises a memory unit 106, a display unit 108 and a power unit 107, wherein the memory unit 106 is connected to the calculation processing unit 105 to record original factory calibration values for pressure, self-calibration information of the blood pressure measuring system, and physiological information described above. In one embodiment, original factory calibration values for pressure can be one set or above. The display unit 108 is connected to the calculation processing unit 105 to display physiological information or blood pressure values; the power unit 107 is electrically connected to the signal detection unit 102, signal transformation unit 103, examining and calibrating unit 109, calculation processing unit 105, memory unit 106 and display unit 108 to supply power.

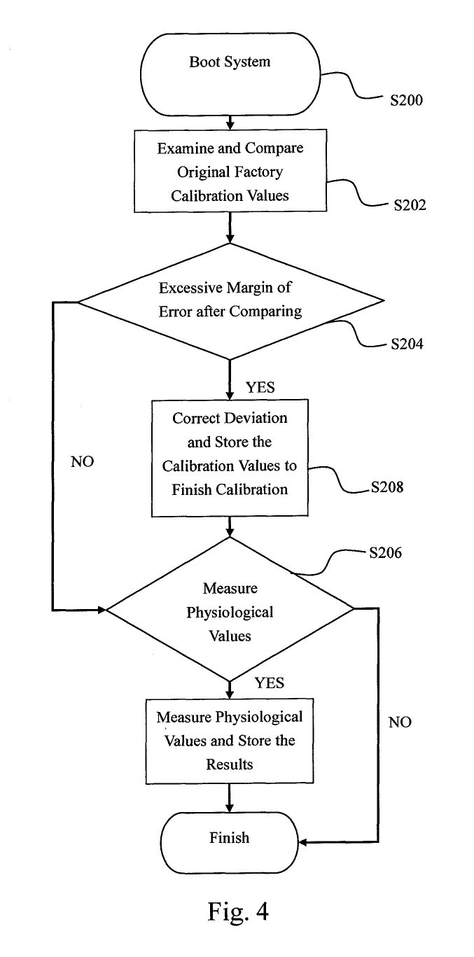

In another embodiment of the present invention, a blood pressure measuring system with automatic self-examination and self-calibration functions 10 further comprises a connecting unit (not shown in the figures), electrically connected to the calculation processing unit 105, for connecting to an external electronic device (not shown in the figures) so as to exchange physiological information or blood pressure values. On the other hand, an external electronic device can supply power to the blood measuring system 10 via the connecting unit.

Wherein the blood pressure measuring system with automatic self-examination and self-calibration functions 10 acquires pressure information to examine and compare when booting the system every time or at specific time. In one embodiment of the present invention, a flow chart of a blood pressure measuring system with automatic self-examination and self-calibration functions shown in FIG. 4. When respondents are in proper posture and ready for measurement by the blood pressure measuring system with automatic self-examination and self-calibration functions 10, execute the examining and calibrating unit to turn on the blood pressure measuring system with automatic self-examination and self-calibration functions 10 (Step S200) and then examine and compare the blood pressure values with the original factory calibration values for pressure (Step S202) to see if the blood pressure is within the margin of error. (Step S204) When the blood pressure value of the blood pressure measuring system with automatic self-examination and self-calibration functions 10 is within the margin of error (i.e. there is no excessive margin of error), it will automatically display that the blood measuring system is normal and users can judge whether the normal measurement mode should proceed (Step 206). If ‘Yes’, physiological value measurement will be performed and measurement results will be stored (Step 208). If ‘No’, the measuring process will be terminated. When the blood pressure value of blood pressure measuring system with automatic self-examination and self-calibration functions exceeds the margin of error, self-calibration mode will be activated to correct deviation and store the calibration values (Step 208). After calibration, the process will return to the previous step to see if the normal measurement mode should be performed. (Step 206).

During the normal measurement mode, pressure information detected by the signal detection unit 102 is blood pressure information. The blood pressure information will be transformed to digital signals via the signal transformation unit 105 and be transmitted to calculation processing unit 105, whereby the measured blood pressure information will be processed. The processing results will be displayed at display unit 108 and stored in the memory unit 106.

In self-calibration mode, a digital blood pressure value is received by the examining and calibrating unit 109 via the signal detection unit 102 and the signal transformation unit 103, which is compared with the original factory calibration value of the memory unit 106 to accomplish self-calibration. FIG. 1 shows a linear diagram of original factory calibration values for blood pressure versus digital signals. For example, blood pressure 0 mmHg is sampled as a calibration point, when a pressure signal exceeds the margin of error in comparison with the original factory calibration value as shown in FIG. 2, the examining and calibrating unit 109 will correct the deviation with the original factory calibration value and write the calibration value to the memory unit 106 to achieve self-calibration.

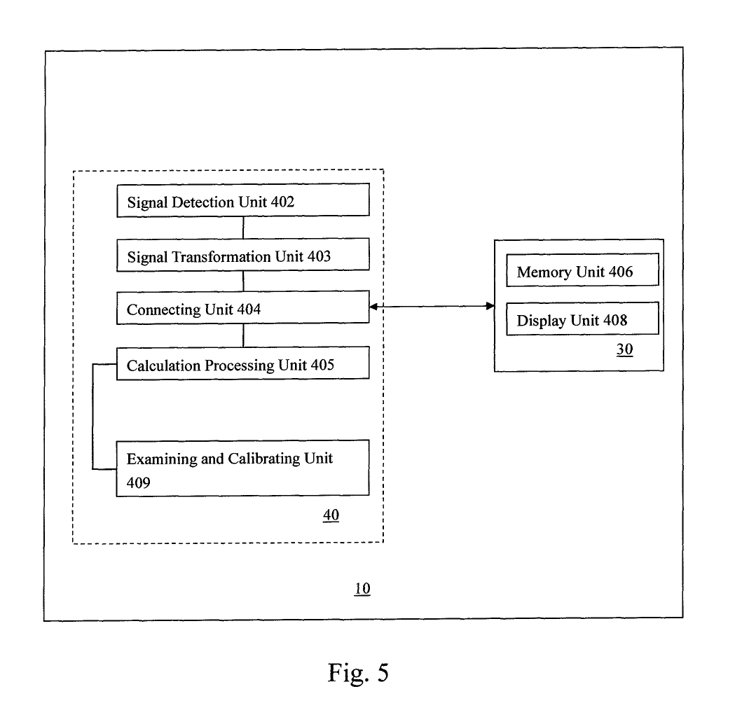

Another embodiment of the present invention shown in FIG. 5, presents a structural schematic diagram of a blood pressure measuring system with automatic self-examination and self-calibration functions 10. A blood pressure measuring system with automatic self-examination and self-calibration functions 10 is composed of a blood pressure measuring device with automatic self-examination and self-calibration functions 40 electrically connected to an external electronic device 30. The blood pressure measuring device with automatic self-examination and self-calibration functions 40 includes: a signal detection unit 402, for detecting an analogous signal for pressure information; a signal transformation unit 403, connected to the signal detection unit 402, for receiving the analogous signal and transforming it to a digital signal of pressure value; a calculation processing unit 405 with built-in original factory calibration values for blood pressure of the blood pressure measuring system 10; and a connecting unit 404 electrically connected to the signal transformation unit 403 and the calculation processing unit 405, for connecting to an external electronic device 30 so as to exchange the digital signal of pressure values and the original factory calibration values for pressure.

Continuing the above description, the blood pressure measuring system with automatic self-examination and self-calibration functions 10 also includes an examining and calibrating unit 409 which is arranged in the blood pressure measuring device with automatic self-examination and self-calibration functions 40 or in the external electronic device 30 for automatically comparing the analogous signal or the digital signal of pressure value with the original factory calibration value for pressure so as to self-calibrate the blood measuring system 10.

In one embodiment, an external electronic device 30 further comprises a memory unit 406 and a display unit 408. The external electronic device 30 calculates physiological information including a blood pressure values according to an analogous signal or a digital signal of pressure value outputted by a connecting unit 404. The memory unit 406 is used for recording one or above sets of original factory calibration values for pressure, self-calibration information of the blood pressure measuring system with automatic self-examination and self-calibration functions 10, and aforementioned physiological information. The display unit 408 is used for displaying physiological information and blood pressure values; the external electronic device supplies power to the blood pressure measuring device 40 via the connecting unit 404.

This blood pressure measuring system with automatic self-examination and self-calibration functions 10 examines and compares pressure information obtained from the detection unit 402 when booting the system every time or at specific time. When respondents are in proper posture and ready for measurement, the external electronic device 30 supplies power to the blood pressure measuring device 40, which can be activated by executing the examining and calibrating unit 109, to examine original factory calibration values for pressure. When a blood pressure value of the blood pressure measuring system with automatic self-examination and self-calibration functions 10 is within the margin of error, it will display that the blood pressure measuring system is normal and a normal measurement mode can be processed; when the blood pressure value of the blood pressure measuring system with automatic self-examination and self-calibration functions 10 exceeds the margin of error, a self-calibration mode will be activated.

In the normal measurement mode, a blood pressure value and physiological values information detected by the signal detection unit 402 are transformed to digital signals by the signal transformation unit 103 and are transmitted to the calculation processing unit 105; all detected blood pressure information will be transmitted to the external electronic device 30 for data processing via the connection unit 404, and be displayed on the display unit 408; the results of processed data will be stored in the memory unit 406. In this embodiment, the external electronic device 30 can be an electrically connected personal computer, a personal digital assistant, a mobile communication device, or a database.

In the self-calibration mode, a digital blood pressure value is received by the examining and calibrating unit 409 via the signal detection unit 402 and signal transformation unit 103, and is compared with the original factory calibration value of the memory unit 106 to achieve self-calibration. FIG. 1 shows a linear diagram of original factory calibration values for blood pressure versus digital signals. For example, blood pressure 0 mmHg is sampled as a calibration point, when the pressure signal exceeds the margin of error in comparison with the original factory calibration value, as shown in FIG. 2, the examining and calibrating unit 109 will correct this deviation with the original factory calibration value to achieve self-calibration. It is noted that the examining and calibrating unit 409 can be built in the blood pressure measuring device with automatic self-examination and self-calibration functions 40 and examine and compare data when booting the system every time or at specific time. Alternatively, the examining and calibrating unit 409 is arranged in the external electronic device 30 in the form of software which can examine and compare data when the blood pressure measuring device with automatic self-examination and self-calibration functions 40 connects to the external electric device 30.

The present invention proposes a blood pressure measuring system with automatic self-examination and self-calibration functions. The significance of the present invention is that, if measuring deviation caused by deformation or distortion of system circuit boards due to long-time use or severe circumstance such as huge temperature difference or huge altitude difference during journey or transportation, the self-calibration mechanism can ensure accuracy for blood pressure measured by users. The proposed electronic sphygmomanometer with self-calibration function according to the present invention, which not only ensures accuracy of the device itself but also provides guarantee to users' health. On the other hand, for hospitals or patients at home, deviation between results detected from the signal detection unit and original factory calibration values may be resulted from distorted circuit boards due to long-time use, and it can cause inaccuracy to measurement results and health crisis to users who rely on values measured and displayed on electronic sphygmomanometers for taking medication or blood pressure control. Therefore, an electronic sphygmomanometer with self-examination function is able to provide patients an accurate indicator for taking medication, prevent serious consequence from miscalculation of blood pressure or physiological values, and saves time and money by skipping original factory calibration process so as to simplify examining and calibrating process and provide high convenience.

In summary, the present invention provides a blood pressure measuring system with automatic self-examination and self-calibration functions, which is capable of measuring and automatically self-examining and self-calibrating. A proper assistance can be provided to patients, elderly people and general users according to their unique requirements. A built-in power unit, a memory unit, or a display unit is not necessary to the blood pressure system with automatic self-examination and self-calibration functions. It has the advantage of low-costs.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the spirit and scope of the appended claims.

What is claimed is:

1. A blood pressure measuring system with automatic self-examination and self-calibration functions comprising:
a signal detection unit, for detecting an analogous signal of pressure information;
a signal transformation unit, connected to the signal detection unit, for receiving the analogous signal and transforming it to a digital signal of pressure value;
an examining and calibrating unit, for automatically comparing at least one of the analogous signal and the digital signal of pressure value with an original factory calibration value so as to self-calibrate for accuracy of the blood measuring system; and
a calculation processing unit, connected to the examining and calibrating unit, the signal detection unit and the signal transformation unit, for calculating physiological information including a blood pressure value according to at least one of the analogous signal and the digital signal of pressure value.

2. The blood pressure measuring system with automatic self-examination and self-calibration functions according to claim 1, further comprising a power unit, which is connected to the signal detection unit, the signal transformation unit, the examining and calibrating unit, and the calculation processing unit, for supplying power.

3. The blood pressure measuring system with automatic self-examination and self-calibration functions according to claim 1, further comprising a memory unit, connected to the calculation processing unit, for recording the original factory calibration value and calibrating at least one of the self-calibration information and the physiological information of the blood pressure measuring system.

4. The blood pressure measuring system with automatic self-examination and self-calibration functions according to claim 1, further comprising a display unit, connected to the calculation processing unit, for displaying at least one of the physiological information and the blood pressure value.

5. The blood pressure measuring system with automatic self-examination and self-calibration functions according to claim 1, further comprising a connecting unit, electrically connected to the calculation processing unit, for connecting an external electronic device so as to exchange the physiological information or the blood pressure information with the external electronic device.

6. The blood pressure measuring system with automatic self-examination and self-calibration functions according to claim 5, wherein the external electronic device supplies power to the blood pressure measuring system via the connecting unit.

7. The blood pressure measuring system with automatic self-examination and self-calibration functions according to claim 1, wherein the blood pressure measuring system is a single-functional blood pressure measuring system or a multifunctional patient monitoring system.

8. A blood pressure measuring device with automatic self-examination and self-calibration functions, electrically connected to an external electronic device, for composing a blood pressure measuring system with automatic self-examination and self-calibration functions; the blood pressure measuring device with automatic self-examination and self-calibration functions comprises:
a signal detection unit, for detecting an analogous signal of pressure information;
a signal transformation unit, connected to the signal detection unit, for receiving the analogous signal and transforming it to a digital signal of pressure value;
a calculation processing unit with a built-in original factory calibration value of a blood pressure measuring system; and
a connecting unit, electrically connected to the signal transformation and the calculation processing unit, for connecting the external electronic device so as to exchange the digital signal of pressure value and the original factory calibration value with the external electronic device.

9. The blood pressure measuring device with automatic self-examination and self-calibration functions according to claim 8, wherein the blood pressure measuring system with automatic self-examination and self-calibration functions further comprises an examining and calibrating unit arranged in the blood pressure measuring device with automatic self-examination and self-calibration functions or in the external electronic device, for automatically comparing at least one of the analogous signal and the digital signal of pressure value with the original factory calibration value so as to self-calibrate for accuracy of the blood pressure measuring system.

10. The blood pressure measuring device with automatic self-examination and self-calibration functions according to claim 8, wherein the external electronic device calculates physiological information including a blood pressure value according to at least one of the analogous signal and the digital signal of pressure value.

11. The blood pressure measuring device with automatic self-examination and self-calibration functions according to claim 10, wherein the external electronic device further comprises a memory unit, for recording the original factory calibration value and calibrating the self-calibration information and the physiological information of the blood pressure measuring system.

12. The blood pressure measuring device with automatic self-examination and self-calibration functions according to claim 10, wherein the external electronic device comprises a display unit, for displaying the physiological information and the blood pressure value.

13. The blood pressure measuring device with automatic self-examination and self-calibration functions according to claim 8, wherein the external electronic device supplies power to the blood pressure measuring device.

14. The blood pressure measuring device with automatic self-examination and self-calibration functions according to claim 8, wherein the blood pressure measuring system is a single-functional blood pressure measuring system or a multifunctional patient monitoring system.
